(19) United States
(12) Patent Application Publication
(10) Pub. No.: US 2017/0273611 A1
(43) Pub. Date: Sep. 28, 2017

(54) SYSTEMS AND METHODS FOR DISCOVERY AND CHARACTERIZATION OF NEUROACTIVE DRUGS

(71) Applicant: The General Hospital Corporation, Boston, MA (US)

(72) Inventors: Patrick L. Purdon, Somerville, MA (US); Emery N. Brown, Brookline, MA (US)

(21) Appl. No.: 15/504,088

(22) PCT Filed: Aug. 24, 2015
(86) PCT No.: PCT/US15/46562

§ 371 Date: Feb. 15, 2017

Related U.S. Application Data
(60) Provisional application No. 62/040,850, filed on Aug. 22, 2014.

(51) Int. Cl. A61B 5/16; G06F 19/00; G06F 19/24; G06F 19/28; A61B 5/04; A61B 5/00

(52) U.S. Cl. CPC A61B 5/16; A61B 5/04001; A61B 5/04004; A61B 5/7235; G06F 19/24; G06F 19/28; G06F 19/706

(57) ABSTRACT

Systems and methods for discovery and characterization of neuroactive drugs are provided. In some aspects, a method for evaluating an effectiveness of a drug administered to a subject includes receiving neurophysiological data acquired from a subject under an administration of a drug, and analyzing the neurophysiological data to generate signatures indicative of brain states induced by the drug. The method also includes correlating the generated signatures with a database comprising information associated with a plurality of drug profiles, and determining, using the information, a molecular activity profile for the drug. The method further includes generating a report indicative of the effectiveness of the drug using the molecular activity profile.

### CROSS-REFERENCE TO RELATED APPLICATIONS

This application claims the benefit of U.S. Provisional Patent Application Ser. No. 62/040,850 filed on Aug. 22, 2014 and entitled “SYSTEMS AND METHODS FOR DISCOVERY AND CHARACTERIZATION OF NEUROACTIVE DRUGS.”

### STATEMENT REGARDING FEDERALLY SPONSORED RESEARCH

This invention was made with government support under DP1-OD003646, DP2-OD006454, and R01-GM104948 awarded by National Institutes of Health. The government has certain rights in the invention.

### BACKGROUND

The field of the invention is related to systems and methods for the characterization and discovery of neuroactive drugs.

Almost 80 years ago, Gibbs, Gibbs and Lenox demonstrated that systematic changes can occur in electroencephalogram (“EEG”) and patient arousal measurements with increasing doses of administered ether or pentobarbital. They recognized the practical application of these observations to be used as measures of the depth of anesthesia. Several subsequent studies reported on the relationship between electroencephalogram activity and the behavioral states of general anesthesia. Faulconer showed in 1949 that a regular progression of the electroencephalogram patterns correlated with the concentration of ether in arterial blood. Linde and colleagues used the spectrum—the decomposition of the electroencephalogram signal into the power in its frequency components—to show that under general anesthesia the electroencephalogram was organized into distinct oscillations at particular frequencies. Bickford and colleagues introduced the compressed spectral array or spectrogram to display the electroencephalogram activity of anesthetized patients over time as a three-dimensional plot (power by frequency versus time). Fleming and Smith devised the density-modulated or density spectral array, the two-dimensional plot of the spectrogram for this same purpose. Levy later suggested using multiple electroencephalogram features to track anesthetic effects.

Since the 1990s, depth-of-anesthesia has been tracked using various indices computed using EEG recordings and behavioral responses to various anesthetic agents obtained using proprietary algorithms. In particular, some indices have been derived by using regression methods relating selected electroencephalogram features to the behavioral responses. One index has been constructed by using classifier methods, deriving a continuum of arousal levels from awake to profound unconsciousness using electroencephalogram recordings categorized visually. Another index related the entropy of an electroencephalogram signal, that is its degree of disorder, to the behavioral response of a patient. These indices are typically computed from the electroencephalogram in near-real-time and displayed on the depth-of-anesthesia monitor as values scaled from 0 to 100, with low values indicating greater depth of anesthesia.

Although the electroencephalogram-based indices have been in use for nearly 20 years, there are several reasons why they are not part of standard anesthesiology practice. First, use of electroencephalogram-based indices does not ensure that awareness under general anesthesia can be prevented. Second, these indices, which have been developed from adult patient cohorts, are less reliable in pediatric populations. Third, because the indices do not relate directly to the neurophysiology of how a specific anesthetic exerts its effects in the brain, they cannot give an accurate picture of the brain's responses to the drugs. Finally, the indices assume that the same index value reflects the same level of unconsciousness for all anesthetics. This assumption is based on the observation that several anesthetics, both intravenous and inhaled agents, eventually induce slowing in the electroencephalogram oscillations at higher doses. The slower oscillations are assumed to indicate a more profound state of general anesthesia.

Two anesthetics whose electroencephalogram responses frequently lead clinicians to doubt index readings are ketamine and nitrous oxide. These agents are commonly associated with faster electroencephalogram oscillations that tend to increase the value of the indices at clinically accepted doses. Higher index values cause concern as to whether the patients are unconscious. At the other extreme, dexmedetomidine can produce profound slow electroencephalogram oscillation and low index values consistent with the patient being profoundly unconscious. However, the patient can be easily aroused from what is a state of sedation rather than unconsciousness. Ambiguities in using electroencephalogram-based indices to define brain states under general anesthesia and sedation arise because different anesthetics act at different molecular targets and neural circuits to create different states of altered arousal, and hence different electroencephalogram signatures. The signatures are readily visible as oscillations in the unprocessed and processed EEG data.

Coordinated action potentials, or spikes, transmitted and received by neurons, are one of the fundamental mechanisms through which information is exchanged in the brain and central nervous system, producing measurable signals indicative of brain activity. As shown in FIG. 1A, neuronal spiking activity generates extracellular electrical potentials, often referred to as local field potentials, which are composed primarily of post-synaptic potentials and neuronal membrane hyperpolarization. The local field potentials produced can then be measured using scalp or intracranial electrodes.

Populations of neurons are thought to play a primary role in coordinating and modulating communication within and among neural circuits. The organization of the pyramidal neurons in the cortex favors the production of large local field potentials because the dendrites of the pyramidal neurons run parallel with each other and perpendicular to the cortical surface. This geometry creates a biophysical transmitting antenna that generates large extracellular currents whose potentials can be readily measured through the skull and scalp. Subcortical regions, such as the thalamus, produce much smaller potentials that are more difficult to detect at the scalp since the electric field decreases in strength as the square of the distance from its source. However, because cortical and subcortical structures are richly interconnected, scalp electroencephalogram patterns reflect the states of both cortical and subcortical structures, as shown in FIG. 1B. Thus, the electroencephalogram provides a window into the brain's oscillatory state.

Over the past several decades, research in molecular pharmacology has provided detailed characterizations of the receptor-level mechanisms for neuroactive drugs used in medical specialties such as anesthesiology, neurology, critical care medicine, and psychiatry. For example, the molecular mechanism of propofol has been well characterized. Propofol binds post-synaptically to GABA_{A} receptors where it induces an inward chloride current which hyperpolarizes the post-synaptic neurons thus leading to inhibition. Since the drug is lipid soluble and GABAergic inhibitory, interneurons are widely distributed throughout the cortex, thalamus, brainstem and spinal cord, inducing sedation through actions at multiple sites (FIG. 2). In the cortex, propofol induces inhibition by enhancing GABA-mediated inhibition of pyramidal neurons. Propofol decreases excitatory inputs from the thalamus to the cortex by enhancing GABAergic inhibition at the thalamic reticular nucleus, a network which provides important inhibitory control of thalamic output to the cortex. Because the thalamus and cortex are highly interconnected, the inhibitory effects of propofol leads not to inactivation of these circuits, but rather to EEG oscillations in the beta and alpha frequency ranges. Propofol also enhances inhibition in the brainstem at the GABAergic projections from the pre-optic area of the hypothalamus to the cholinergic, monoaminergic and orexinergic arousal centers. Decreasing excitatory inputs from the thalamus and the brainstem to the cortex enhances hyperpolarization of cortical pyramidal neurons, an effect which favors the appearance of slow and delta frequency. Other drugs, can act according to different molecular mechanisms, producing readily distinguishable EEG signatures. For example, ketamine acts primarily by binding to NMDA receptors in the brain and spinal cord, while dexmedetomidine alters arousal primarily through its actions on pre-synaptic α2adrenergic receptors on neurons projecting from the locus cureleus.

The profile of molecular receptors at which various drugs act necessarily relates to the drug's behavioral and clinical effects. However, characterizing how actions at the molecular level translate to higher-level neural circuit, system, network, and behavioral effects is one of the most challenging problems in modern medicine. In addition, although drugs have been developed to act at specific receptor types, in practice most drugs have affinities for a number of different receptors. The relative importance of a drug's diverse molecular receptor actions is difficult to estimate in vitro, because in vitro drug concentrations that are physiologically equivalent to clinical doses are hard to establish. Also, relative contributions of different receptor actions to neuronal, circuit, system, network, and behavioral levels are even more difficult to characterize.

Thus, a critical priority for improved discovery and development of neuroactive drugs is to develop systems and methods for characterizing multiscale drug neurophysiology, linking actions at the molecular level to neurophysiological dynamics at the neuronal, circuit, system, and network levels, as well as behavior and clinical outcomes.

### SUMMARY

The present disclosure describes systems and methods for use in characterization and discovery of neuroactive drugs. In particular, the present disclosure recognizes that electroencephalogram (“EEG”), behavioral, and other brain signatures are reflective of the various molecular mechanisms by which anesthetic and other neuroactive drugs affect brain activity. Using neurophysiological measurements across various scales of brain organization, from neurons to neural circuits, systems and networks, as well as behavior and clinical outcome measurements, information indicative of molecular-level actions, as well as other information characterizing administered neuroactive drugs, can be extracted.

In accordance with one aspect of the disclosure, a system for evaluating an effectiveness of one or more drugs administered to a subject is provided. The system includes an input configured to receive neurophysiological data acquired from a subject, and a processor programmed to at least analyze the neurophysiological data to generate signatures indicative of brain states induced by one or more drugs administered to the subject. The processor is also programmed to correlate the generated signatures with a database comprising information associated with a plurality of drug profiles, and determine, using the information, a molecular activity profile for the one or more drugs. The process is further programmed to generate, using the molecular activity profile, a report indicative of the effectiveness of the one or more drugs. The system also includes an output for displaying the report.

In accordance with another aspect of the disclosure, a method for evaluating an effectiveness of a drug administered to a subject is provided. The method includes receiving neurophysiological data acquired from a subject under an administration of a drug, and analyzing the neurophysiological data to generate signatures indicative of brain states induced by the drug. The method also includes correlating the generated signatures with a database comprising information associated with a plurality of drug profiles, and determining, using the information, a molecular activity profile for the drug. The method further includes generating a report indicative of the effectiveness of the drug using the molecular activity profile.

In accordance with yet another aspect of the disclosure, a method for characterizing a neuroactive drug administered to a subject is provided. The method includes acquiring neurophysiological data using one or more sensors arranged about a subject having received a neuroactive drug, and generating, using the neurophysiological data, signatures indicative of brain states induced by the neuroactive drug. The method also includes identifying, using the generated signatures, molecular receptor actions associated with the induced brain states, and generating a report characterizing the neuroactive drug using the identified molecular receptor actions.

The foregoing and other aspects and advantages of the invention will appear from the following description. In the description, reference is made to the accompanying drawings that form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1A is a schematic diagram illustrating how cortical and subcortical local field oscillations are generated.

FIG. 1B is a graphical illustration showing example waveforms of neural spiking and local field oscillations.

FIG. 2 is a schematic showing the neurophysiological mechanisms of propofol in the brain.

FIG. 3 is a graphical illustration showing different neurophysiological mechanisms and respective signatures for different neuroactive drugs.

FIG. 4A is a schematic diagram showing an example system, in accordance with aspects of the present disclosure.

FIG. 4B is a schematic diagram showing an example monitoring system, in accordance with aspects of the present disclosure.

FIG. 4C a schematic diagram showing an example sensor assembly, in accordance with aspects of the present disclosure.

FIG. 5 is a flowchart setting forth steps of a process, in accordance with aspects of the present disclosure.

FIG. 6 is a flowchart setting forth steps of a process, in accordance with aspects of the present disclosure.

FIG. 7 is a graphical illustration comparing signatures of induced brain states between sevoflurane and propofol.

FIG. 8A is a schematic showing an illustration of electroencephalogram channels and example coherence measurement using three generated signals.

FIG. 8B is a schematic illustrating spectrograms and coherograms generated using the signals of FIG. 8A.

FIG. 9A is an example group spectrogram for patients subjected to propofol general anesthesia.

FIG. 9B is an example group spectrogram for patients subjected to sevoflurane general anesthesia.

FIG. 9C is a graphical example showing a comparison of spectral power distributions for sevoflurane and propofol across a range of frequencies.

FIG. 10A is a group coherogram for patients subjected to propofol general anesthesia.

FIG. 10B is a group coherogram for patients subjected to sevoflurane general anesthesia.

FIG. 10C is a graphical example showing a comparison of coherence for sevoflurane and propofol across a range of frequencies.

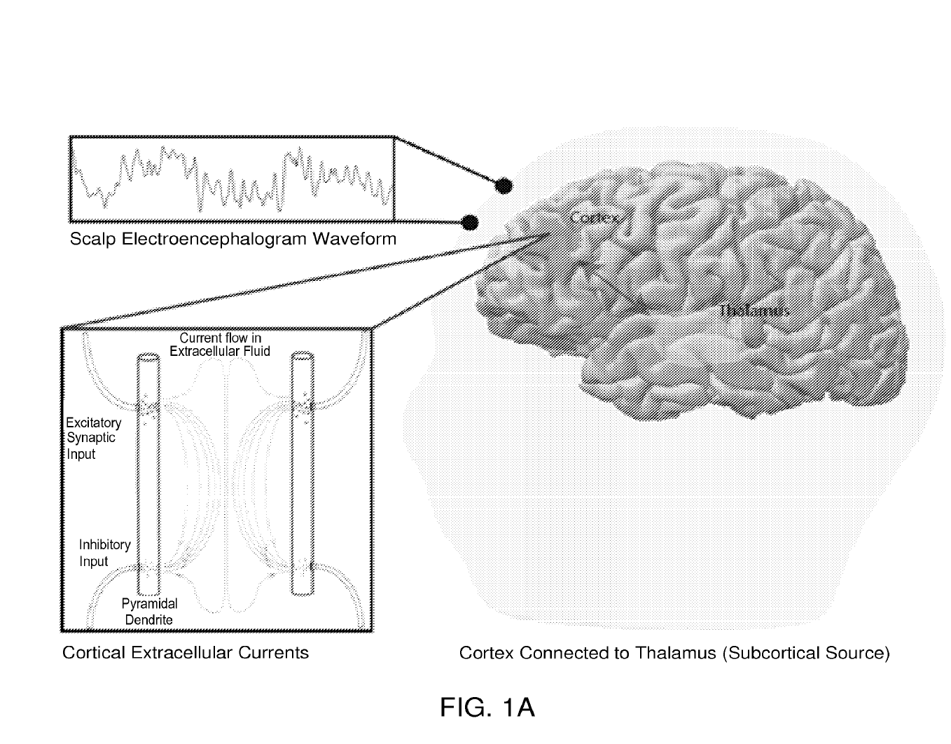

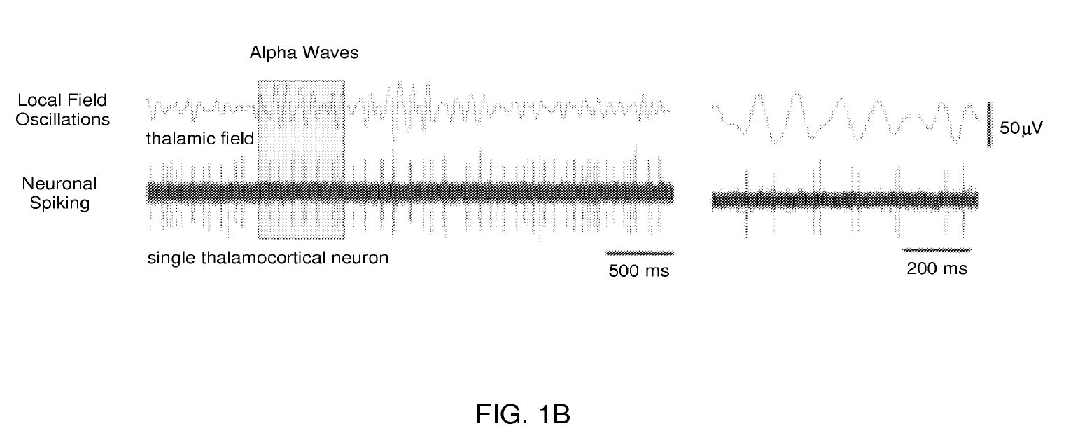

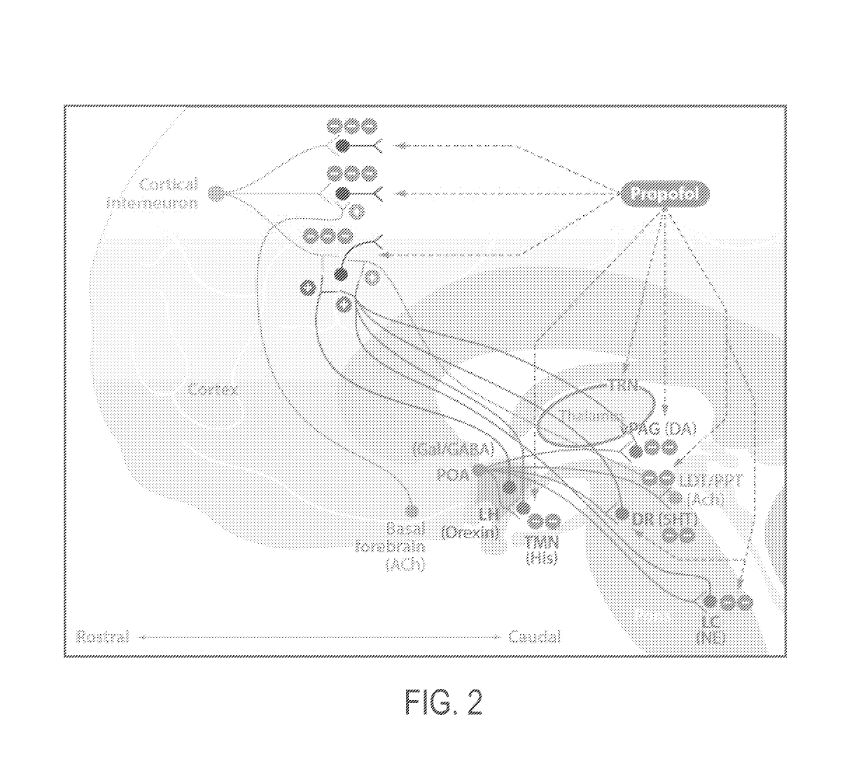

### DETAILED DESCRIPTION

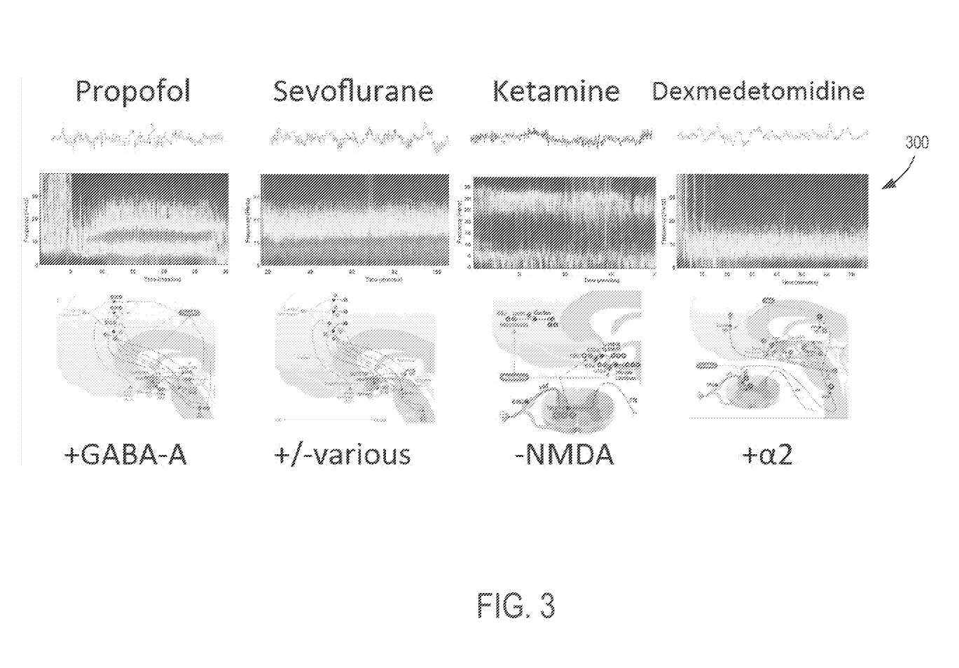

The present disclosure describes systems and methods for use in characterizing one or more neuroactive drugs. As will be described, neurophysiological measurements, such as electroencephalogram (“EEG”), under administration of anesthetics or other neuroactive drugs produce particular signatures indicative of the various molecular mechanisms by which various brain states are achieved. For example, as shown in FIG. 3, distinct spectrogram signatures 300, representing the time variation of spectral content associated with measured brain signals, are readily apparent between propofol, sevofluarane, ketamine and dexmedetomidine. As shown, the molecular mechanisms dictating the specific actions affecting brain states can also vary across the different neuroactive drugs.

Therefore, it is a discovery of the present invention that brain, behavioral, and other physiological signatures and information associated with known neuroactive drugs can be utilized to characterize one or more administered drug. Using systems and methods provided, various analytical analyses can be performed on acquired physiological data, to determine the effects and potential side-effects of an unknown drug. In some aspects, relationships between molecular receptor activity profiles and measurable neurophysiological dynamics may be determined. For instance, neural circuit modeling may be performed to map molecular-level actions onto systems-level dynamics. As will be appreciated from descriptions below, the approaches of the present disclosure are beneficial to many areas of drug characterization and discovery, including drug candidate screening, adjustment of particular prototype compounds, and so forth.

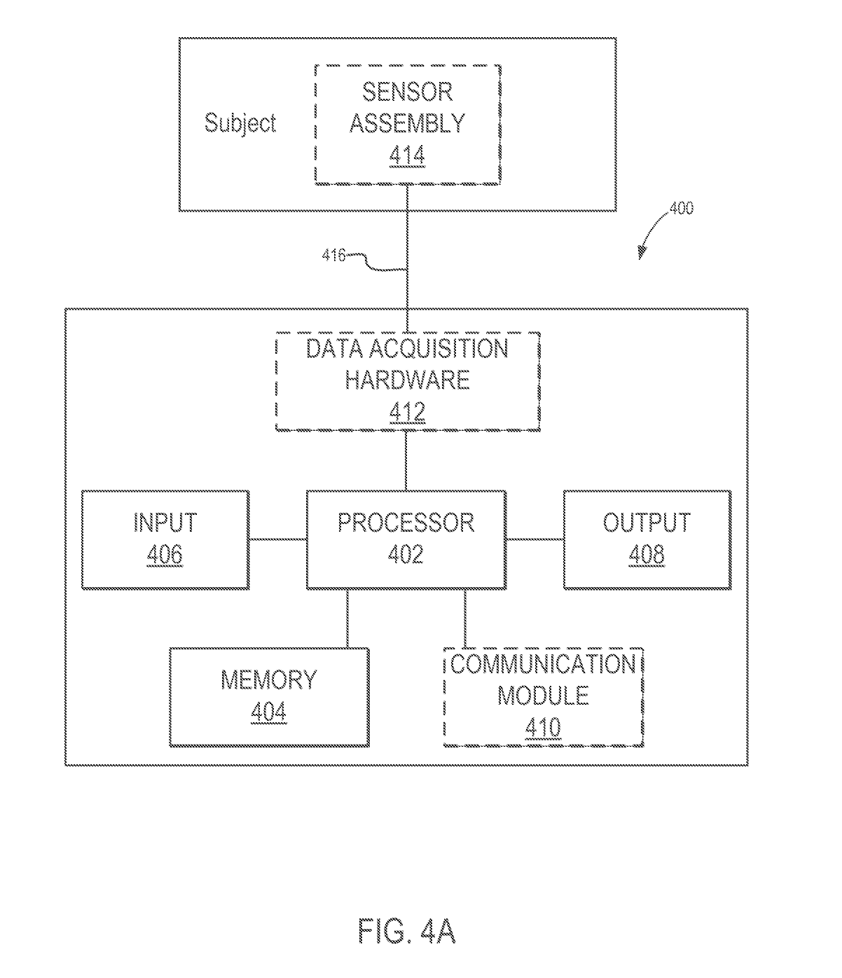

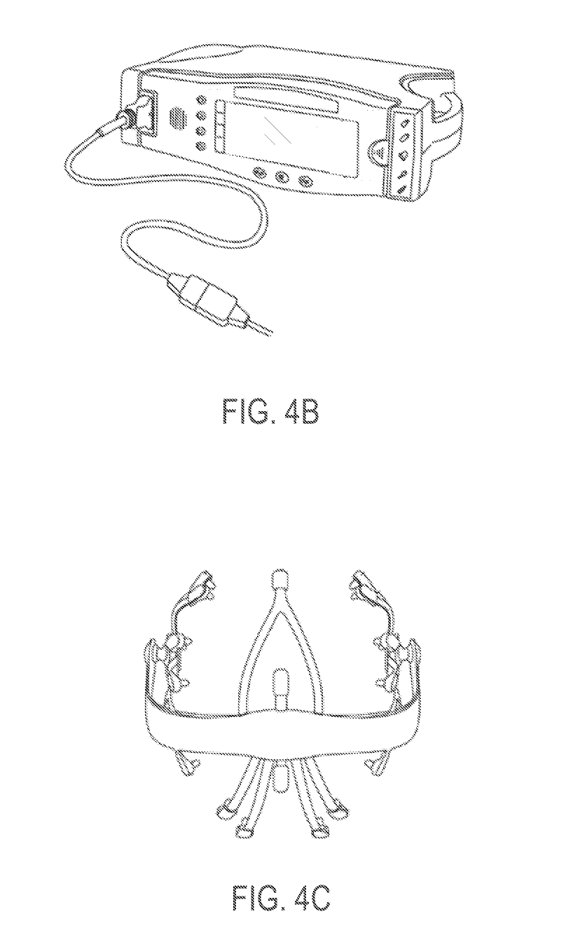

Turning to FIG. 4A, a schematic diagram of an example system 400 for use in accordance with aspects of the present disclosure. In general, system 400 may be any computing device, apparatus or system capable of a wide range of functionality, integrating a variety of software and hardware capabilities. As shown in FIG. 4A, in some configurations, the system 400 includes a processor 402, a memory 404, an input 406, and an output 408. The system 400 may operate either independently or as part of, or in collaboration with any computer, system, device, machine, mainframe, database, server or network. In some aspects, the system 400 may be a portable or wearable device or apparatus, for example in the form of a mobile device, tablet, smartphone, smartwatch, and the like. Alternatively, the system 400 may be configured to communicate with such portable or wearable device or apparatus, for example, via a communication module 410, via Bluetooth or other wireless communication protocol.

In some embodiments, the system 400 may be a monitoring system, as shown in the example of FIG. 4B, and include data acquisition hardware 412 in communication with a sensor assembly 414. In particular, the sensor assembly 414 may include any number of active and/or passive sensing elements, and may be configured to measure a variety of signals associated with a monitored subject, including brain activity, muscle activity, respiration activity, cardiac activity, eye movement, galvanic skin response, blood oxygenation, as well as motion, pressure, temperature, force, sound, flow, and so forth. Non-limiting examples include EEG sensors, electromyography (“EMG”) sensors, cardiac sensors, respiratory sensors and other sensors. In some configurations, the sensor assembly 414 may be in the form of a device to be worn by the subject, as shown in the example of FIG. 4C.

For clarity, a single block is used to illustrate the sensor assembly 414 shown in FIG. 4A. However, it should be understood that the sensor assembly 414 shown can include more than one sensing element or sensing element types configured to capture a variety of neurophysiological signals. For example, sensors in the sensor assembly 414 can include electrical sensors, oxygenation sensors, galvanic skin response sensors, respiration sensors, muscle activity sensors, pressure sensors, force sensors, temperature sensors, air flow sensors, and so forth, and any combinations thereof. In addition, sensors may be placed at multiple locations about a monitored subject, including, but not limited to, the scalp, face, nose, chin, skin, chest, limbs, fingers, and so on, as well as within the subject's anatomy via intra-cranial probes.

Neurophysiological signals generated by the sensor assembly 414 may then transmitted to the system 400 over a cable or other communication link 416 or medium, such as wireless communication link, digitized using the analog/digital converter (not shown) associated with the data acquisition hardware 412, and processed using one or more processor 402. In some embodiments of the system 400 shown in FIG. 4A, all of the hardware used to receive and process signals from the sensors are housed within the same housing. In other embodiments, some of the hardware used to receive and process signals is housed within a separate housing. In addition, the system 400 of certain embodiments includes hardware, software, or both hardware and software, whether in one housing or multiple housings, used to receive and process the signals transmitted by the sensors.

The processor 402 may configured to carry out any number of steps for operating the system 400. In addition, the processor 402 may be programmed to process and analyze neurophysiological data acquired from a subject for characterizing one or more neuroactive drugs. In some aspects, neurophysiological data may be provided intermittently or in real time via the data acquisition hardware 412, or retrieved from the memory 404, a database, or other storage location. Alternatively, the data may be received by the process 402 via input 406. The processor 402 may also be configured to receive an indication from a user via input 406. For example, a user may specify the clinical state targeted, such as sedation, general anesthesia, recovery from depression, suppression of epileptic activity, and so forth. Such indication, along with information associated with a monitored subject, such as age, medical condition, and so forth, may be utilized in analyses for characterizing targeted drugs, as described below.

In some aspects, the processor 402 may configured to perform signal conditioning or pre-processing, such as scaling, amplifying, or selecting desirable signals, or filtering interfering or undesirable signals. In addition, the processor 402 may be configured to assemble the acquired neurophysiological data in various forms suitable for identifying signatures indicative of brain states induced using one or more neuroactive drugs, reflecting neurodynamics at various scales. In particular, the processor 402 may be configured to generate spectral, waveform, and other representations. For instance, the processor 402 assemble a time frequency representation of the acquired neurophysiological data in the form of spectrograms using a multitaper technique.

In accordance with aspects of the present disclosure, the processor 402 may also be configured to generate, using the neurophysiological data, spatial and temporal signatures indicative of brain states induced by one or more administered neuroactive drug. Non-limiting examples of brain states can include awake states, loss of consciousness states, levels or states of consciousness, sleep states, wakefulness states, sedation states, burst suppression states, cognitive states, emotional states, and so forth. As such, the processor 402 may perform a number of analyses to generate the signatures, including waveform analyses, spectral analysis, coherence analyses, amplitude analyses, phase analyses, phase-amplitude modulation analyses, synchrony analyses, statistical analysis, behavioral analyses, and so forth, using neurophysiological signals as well as any information related to the subject and neuroactive drug administered.

By way of example, reference is made to analyses described in application PCT/US2014/035178 entitled “System and method for monitoring anesthesia and sedation using measures of brain coherence and synchrony”,” incorporated herein by reference, in its entirety. As another example, analysis methods described in Purdon et al. (“Electroencephalogram signatures of loss and recovery of consciousness from propofol,” Proceedings of the National Academy of Sciences, 2013) may also be used, incorporated herein by reference, in its entirety. In addition, in some aspects, the processor 402 may also be configured to perform a neural circuit modeling to map molecular level actions onto circuit or system-level dynamics. As an example of this neural circuit modeling process, modeling and simulation methods described in Ching et al. (“Thalamocortical model for a propofol-induced alpha-rhythm associated with loss of consciousness,” Proceedings of the National Academy of Sciences, 2010) may also be used, incorporated herein by reference, in its entirety.

As mentioned, signatures indicative of brain states induced by one or more administered neuroactive drugs can be in the form of waveforms, spectrograms, power spectra, and so forth, reflecting neurophysiological dynamics at various scales. In some aspects, the signatures may include brain maps, for instance, reflecting spatial power distribution across locations of the brain for various frequencies, or frequency ranges, such as alpha, beta, gamma, delta and low frequency ranges. Such brain maps may also reflect the spatial distribution of coherence and/or synchrony of signals at various frequencies, or frequency ranges. Using such signatures, the processor 402 may be configured to provide a characterization of one or more administered drugs using measured neurophysiological data. In particular, the processor 402 may correlate generated signatures with a database or library that includes information and measurement signatures for a number of drug profiles, including information regarding behavioral and clinical effects of the drugs, as well as information regarding molecular-level receptor activity. The drug profiles in the database can also be categorized in dependence of different subject characteristics, whether animal or human, including age, medical condition, neurophysiological recording location, and so forth, as well as drug administration characteristics, such as drug dose, drug timing, and so forth.

In some aspects, the processor 402 may also be configured to determine, using information in the database as described above, a molecular activity profile for the one or more administered drugs from measured neurophysiological data. In some aspects, the molecular activity profile may include information on particular brain circuits within which the drug is likely to act. In applications where the administered drug is unknown, the determined molecular activity profile, along with other information, may be used to characterize or identify the drug, specifying, for instance, dose response, clinical outcome, or possible side effects. In some aspects, a determined molecular activity profile may reflect molecular-level actions, including receptor activities, as well as a hierarchy of molecular affinities.

In some aspects, the processor 402 may be configured to generate simulation data for the one or more drugs based by performing a simulation using the molecular activity profiles determined. This may be used to validate the accuracy of a drug characterization by performing a comparison between the simulation data and the acquired neurophysiological data. The simulation could be based, for instance, on neurophysiological models employing realistic representations of neural circuit architectures within different interconnected structures such as the thalamus, cerebral cortex, and brainstem, for instance. The simulation could also employ realistic representations of neurophysiological dynamics, for instance, using Hodkin-Huxley models of different ion channels and receptors. As such, based on the characterization performed, the processor 402 may generate information regarding an effectiveness of the analyzed drug(s). Such information, may help inform a modification of the drug(s) or components thereof. In some applications, such information may be helpful in selecting drug candidates for achieving one or more targeted clinical states.

The processor 102 may then generate and provide a report either intermittently, or in real time, via output 408, which may include a display and/or speaker, or other output elements. The report may be any form, and include any information, including information related to acquired and processed neurophysiological and behavioral data, for instance as waveforms or time series traces, time frequency representations, power spectra, response curves, spectrograms, brain maps, and so on. In some aspects, the report may include information regarding a characterized or unknown drug(s). For example, the report may include a molecular activity profile or an effectiveness of one or more administered drug. The report may also include information regarding one or more determined brain states.

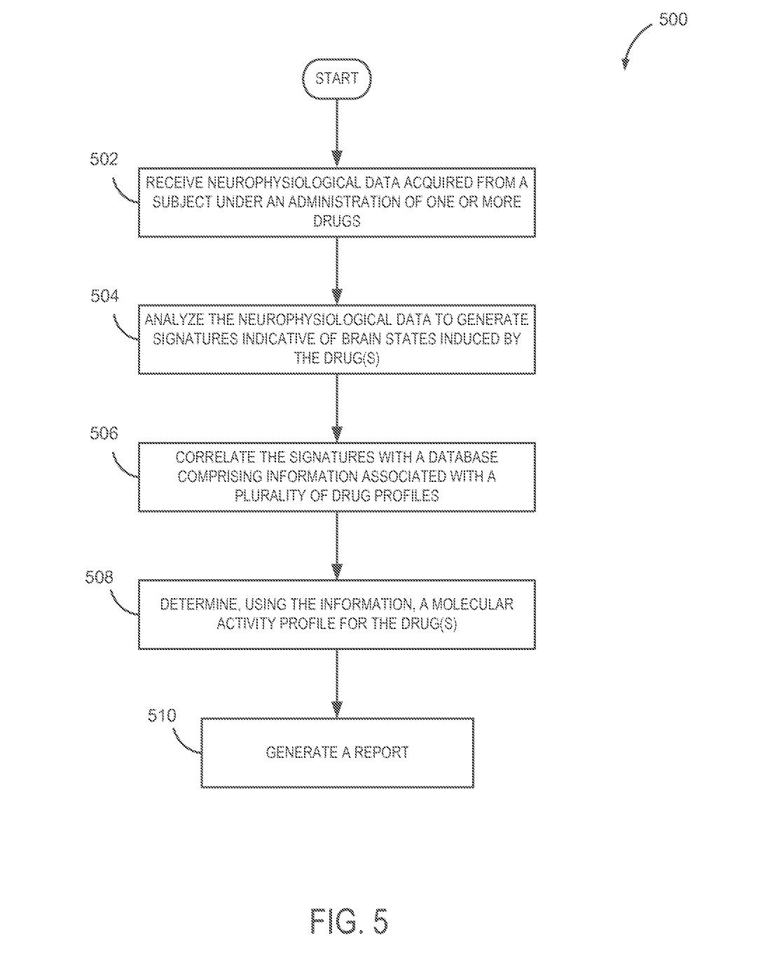

Turning to FIG. 5, steps of a process 500 for evaluating an effectiveness of one or more drug administered drugs are shown. Specifically, process 500 be carried out using any suitable computing devices or systems, such as systems described with respect to FIGS. 4A-C. At process block 502 neurophysiological data acquired from a subject under an administration of a drug may be received. In some aspects, the neurophysiological data using one or more sensors arranged about the subject. Non-limiting examples of neurophysiological data include EEG data, electromyography (“EMG”) data, behavioral data, respiratory data, blood flow data, cardiac data, and galvanic skin response data, and so forth.

At process block 504, analyses may be performed using the acquired data, to generate signatures indicative of brain states induced by the administered drug(s). As described, such signatures can be in the form of waveforms, spectrograms, power spectra, brain maps and so forth, reflecting neurophysiological dynamics at various scales and over various neural circuits. As described, this may include assembling the neurophysiological data into various representations, such as a frequency representation using a multitaper technique, and performing waveform analyses, spectral analyses, coherence analyses, phase analyses, amplitude analyses, synchrony analyses, statistical analyses, and so forth. Then, at process block 506, a correlation may be performed using the signatures generated. Specifically, the generated signatures may be correlated with information associated with a plurality of drug profiles stored in a database or other storage location.

Then at process block 508 a drug characterization can be performed. In some aspects, a molecular activity profile for the drug(s) may be determined using information found in the database and determined signatures. In addition, brain states of the analyzed subject may also be determined. Example brain states can include states of sedation, anesthesia, sleep, depression, cognitive impairment, unconsciousness, wake, arousal and so forth. In some aspects, an indication of one or more clinical or brain states targeted by the administered drugs may also provided and utilized in characterizing the drug(s). Using the determined characteristics, such as a molecular activity profile, or signatures, an unknown drug can be identified.

Then at process block 510, a report may be generated, in any form. In some aspects, the report may include information regarding a characterized or unknown drug(s). For example, the report may include a molecular activity profile or an effectiveness of one or more administered drug. The report may also include information regarding one or more determined brain states.

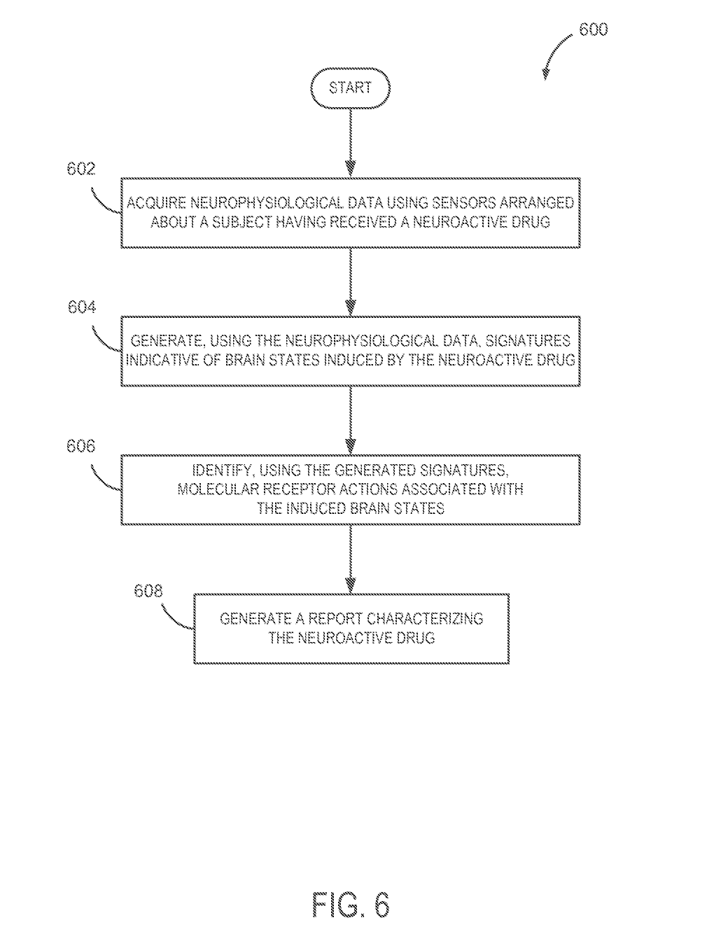

Turning to FIG. 6, steps of another process 600, in accordance with aspects of the present disclosure, are shown. The process 600 may begin at process block 602 with acquiring neurophysiological data using one or more sensors arranged about a subject having received a neuroactive drug. As described, the sensors can be invasive or non-invasive, and include electrical sensors, oxygenation sensors, galvanic skin response sensors, respiration sensors, muscle activity sensors, pressure sensors, force sensors, temperature sensors, air flow sensors, and so forth, and any combinations thereof. In particular, the acquired neurophysiological data may be associated with various neurons, neural circuits, systems or networks. Alternatively, the neurophysiological data may be retrieved from a data storage location, or a memory at process block 602.

Then, at process block 604, signatures indicative of brain states induced by the neuroactive drug may be generated by performing a number of analyses on the acquired neurophysiological data, as described. The generated signatures may then be utilized to characterize the nature of the administered neuroactive drugs. For instance, molecular receptor actions may be identified from the generated signatures, as indicated by process block 606. In addition, a dominant receptor affinity based on a dose of the neuroactive drug may also be identified. As described, this may include comparing generated signatures with a database that includes information associated with a plurality of known drug profiles. In some aspects, information associated a patient profile, as well as administration characteristics of the neuroactive drugs) may be received.

Then, at process block 608, a report characterizing the analyzed neuroactive drugs. As described, the report may include a variety of information, including molecular activity profiles, or molecular receptor actions, such as a dominant receptor action, associated with the induced brain states. Such information may be utilized, for example, when selecting drug candidates for a targeted clinical state, or when determining a modification to one or more drug compounds. As described, in some aspects, such information may be utilized to generate simulation data in order to perform a verification of the identified drug characteristics.

Specific examples are provided below, illustrative of the above-described systems and methods. These examples are offered for illustrative purposes only, and are not intended to limit the scope of the present disclosure in any way. Indeed, various modifications of the disclosure in addition to those shown and described herein will become apparent to those skilled in the art from the foregoing description and the following examples, and fall within the scope of the appended claims.

### Example

Previously, neural mechanisms of anesthetic vapors have not been studied in depth. However, modeling and experimental studies on the intravenous anesthetic propofol indicate that potentiation of γ-Aminobutyric acid receptors leads to a state of thalamocortical synchrony, observed as coherent frontal alpha oscillations, associated with unconsciousness. Sevoflurane, an ether derivative, also potentiates γ-Aminobutyric acid receptors (“GABA”), as well as a number of other receptors such as N-methyl-D-aspartate receptor (“NMDA”). However, in humans, sevoflurane-induced coherent frontal alpha oscillations have not been well detailed.

To investigate the electroencephalogram dynamics induced by sevoflurane, age and gender matched patients were selected in which sevoflurane (n=30) or propofol (n=30) were used as the sole agent for maintenance of general anesthesia during routine surgery. Then, the electroencephalogram (“EEG”) signatures of sevoflurane were compared to those to propofol using time-varying spectral and coherence methods. As will be described, sevoflurane general anesthesia was characterized by alpha oscillations with maximum power and coherence at approximately 10 Hz, (mean±std; peak power, 4.3 dB±3.5; peak coherence, 0.73±0.1). These alpha oscillations were similar to those observed during propofol general anesthesia, which also had maximum power and coherence at approximately 10 Hz (peak power, 2.1 dB±4.3; peak coherence, 0.71±0.1). However, sevoflurane also exhibited a distinct theta coherence signature (peak frequency, 4.9 Hz±0.6; peak coherence, 0.58±0.1). In addition, slow oscillations were observed in both cases, with no significant difference in power or coherence. These results indicate that sevoflurane, like propofol, induces coherent frontal alpha oscillations and slow oscillations in humans to sustain the anesthesia-induced unconscious state. As such, there is a shared molecular and systems-level mechanism for the unconscious state induced by these drugs.

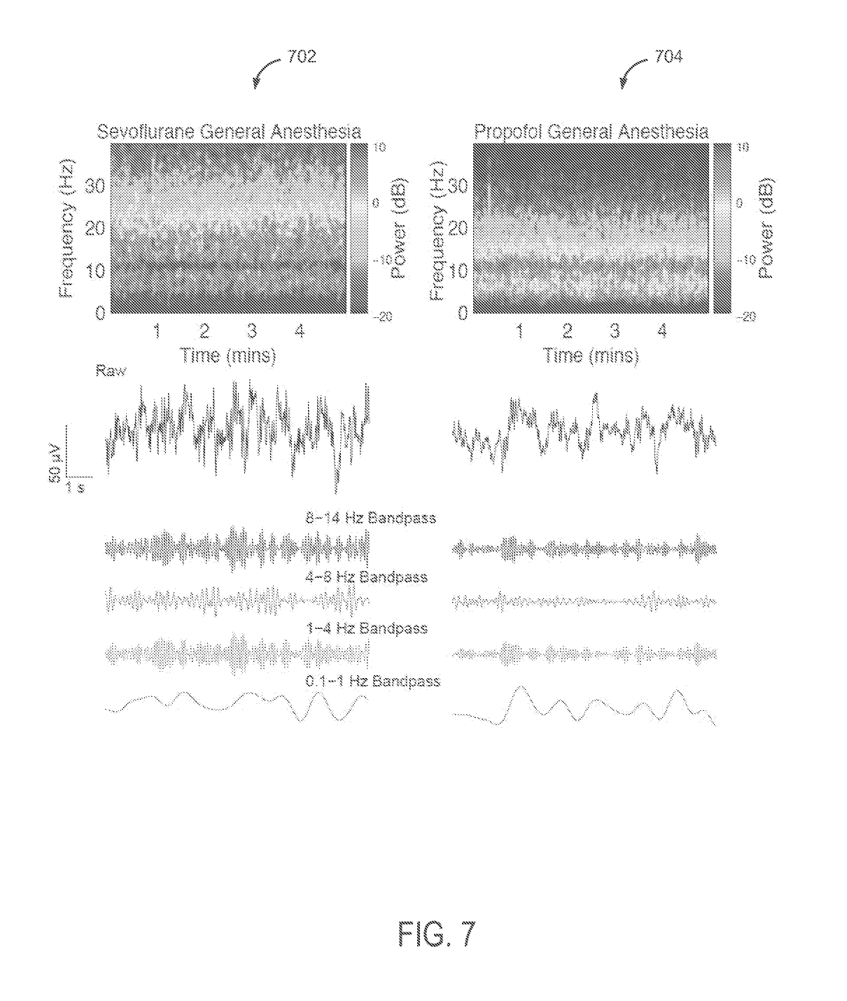

Sevoflurane is an anesthetic agent with a rapid induction, emergence and recovery profile. Evidence suggests that sevoflurane, similar to other ether derivatives in clinical use, exerts its physiological and behavioral effects by binding at multiple targets in the brain and spinal cord. Action at these targets includes potentiation of γ-Aminobutyric acid (GABA_{A}), glycine and two-pore potassium channels; and inhibition of voltage gated potassium, N-methyl-D-aspartate, muscarinic and nicotinic acetylcholine, serotonin, and α-Amino-3-hydroxy-5-methyl-4-isoxazolepropionic acid channels. Despite detailed characterizations of the molecular and cellular pharmacology of anesthetics, the neural circuit-level mechanisms of general anesthesia-induced unconsciousness are still being actively investigated. Extensive work has helped propose neural circuit mechanisms to the electroencephalogram patterns of propofol (2,6-di-isopropylphenol). Clinically, sevoflurane was observed to induce stereotypical changes in the electroencephalogram that appear similar to those propofol, as shown in FIG. 7. Hence, comparing the electroencephalogram dynamics induced by sevoflurane to propofol can provide insights into the neural circuit mechanism through which sevoflurane and other ether derivatives induce unconsciousness.

Propofol primarily acts at GABA_{A} receptors throughout the brain and spinal cord to enhance inhibition. It also potentiates glycine receptors, and provides inhibition to voltage gated potassium, acetylcholine, α-Amino-3-hydroxy-5-methyl-4-isoxazolepropionic and kainate channels amongst others. Unconsciousness under propofol is characterized in the electroencephalogram by alpha (8-12 Hz) oscillations that are coherent across the frontal cortex, delta (1-4 Hz) oscillations, and high amplitude incoherent slow (0.1-1 Hz) oscillations. Intracortical recordings during propofol-induced unconsciousness suggest that local and long range cortical communication are impeded by spatially incoherent slow oscillations that exhibit phase-limited spiking.

Analysis of the scalp electroencephalogram, a readily accessible measure of the average activity in large populations of cortical neurons, has established that propofol induces synchronous frontal alpha oscillations. Biophysical modeling provides further evidence that propofol induces coherent alpha activity by increasing GABAA conductance and decay time. This increase in GABA_{A} conductance facilitates involvement of the thalamus in a highly coherent thalamocortical alpha oscillation loop. This pathologically coherent frontal alpha oscillation pattern reduces the dimensionality of the thalamocortical network, reducing the ability of the thalamus to project and coordinate exogenous inputs to the neocortex. Coherent alpha oscillations have also been identified in animal studies of the inhaled anesthetics during unconsciousness. However, human studies examining this inhaled anesthesia-induced electroencephalogram dynamics are limited. Given that both sevoflurane and propofol are known to act at GABA_{A} receptors, it is possible that comparing the electroencephalogram patterns elicited by sevoflurane to those elicited by propofol can provide insights into the neural circuit mechanisms of sevoflurane. Given a similar GABAergic mechanism of action, it was hypothesized herein that the spectral and coherence features of sevoflurane general anesthesia would be similar to propofol general anesthesia. That is, at surgical anesthetic depth, there would be a predominance of large amplitude slow, delta, and coherent alpha oscillations.

To explore these hypotheses, an observational study was performed to record intraoperative frontal electroencephalogram in 30 patients undergoing general anesthesia with sevoflurane or propofol as the primary maintenance agent. Electroencephalogram dynamics during sevoflurane and propofol general anesthesia were compared using time varying spectral and coherence methods, as described below.

A database of anesthesia and electroencephalogram recordings and identified age and gender matched patients in which sevoflurane (n=30) or propofol (n=30) were used as the sole hypnotic agent for maintenance of general anesthesia during routine surgery. Table 1 summarizes the patient characteristics while Table 2 summarizes the end tidal sevoflurane vapor concentration and propofol infusion rates used during the maintenance phases of the electroencephalogram epochs selected. Table 3 provides additional information on co-administered medications.

TABLE 1
Characteristics of Patients Studied

| | Sevoflurane (n = 30) | | Propofol (n = 30) | |
|---|---|---|---|---|
| Age (years), mean (±SD) | 43 | (17) | 45 | (16) |
| Sex (male), n (%) | 11 | (36.7) | 11 | (36.7) |
| Weight (kg), mean (±SD) | 83 | (23) | 81 | (18) |
| BMI (kg/m²), mean (±SD) | 30 | (9) | 30 | (7) |
| Surgery type, n (%) | | | | |
| General | 16 | (53.3) | 17 | (56.7) |
| Gynecologic | 3 | (10.0) | 2 | (6.7) |
| Orthopedic | 3 | (10.0) | 1 | (3.3) |
| Plastic | 4 | (13.3) | 5 | (16.7) |
| Thoracic | 0 | (0) | 1 | (3.3) |
| Urologic | 4 | (13.3) | 4 | (13.3) |
| Length of Surgery (minutes), | | | | |
| mean (±SD) | 126 | (72) | 126 | (109) |

BMI, body mass index; kg, kilogram; m, meter; SD, standard deviation.

TABLE 2
General Anesthesia Induction and Maintenance Agents

| Sevoflurane (n = 30) | | Propofol (n = 30) | |
|---|---|---|---|
| Induction agent (mg), | Propofol | Induction | Propofol |
| mean (±SD) | (n = 28) | agent (mg), | (n = 30) |
| | 205 (66) | mean (±SD) | 198.3 (44) |
| | Methohexital | | |
| | (n = 1) 250 | | |
| | Etomidate | | |
| | (n = 1) 30 | | |
| Maintenance | 2.21 (0.44) | Maintenance | 117.2 (26) |
| sevoflurane* | | propofol* | |
| (% inspired), | | (mcg/kg/min), | |
| mean (±SD) | | mean (±SD) | |

*Maintenance anesthetic during the selected epoch.
SD, standard deviation.

Frontal electroencephalogram data were recorded using the Sedline brain function monitor (Masimo Corporation, Irvine Calif.). The electroencephalogram data were recorded with a pre-amplifier bandwidth of 0.5 to 92 Hz, sampling rate of 250 Hz, with 16-bit, 29 nV resolution. The standard Sedline Sedtrace electrode array records from electrodes located approximately at positions Fp1, Fp2, F7, and F8, with ground electrode at Fpz, and reference electrode approximately 1 cm above Fpz. Electrode impedance was less than 5 kΩ in each channel. An investigator experienced in reading the electroencephalogram (O.A.) visually inspected the data from each patient and selected electroencephalogram data free of noise and artifacts for analysis.

Electroencephalogram data segments were selected using information from the electronic anesthesia record. For each patient, 5-minute EEG segments representing the maintenance phase of general anesthesia during surgery were carefully selected. The data was selected from a time period after the initial induction bolus of an intravenous hypnotic and while the maintenance agent was stable. These data have not been reported upon in previous publications.

The power spectral density, also referred to as the power spectrum or spectrum, quantifies the frequency distribution of energy or power within a signal. For example, FIG. 7 shows representative electroencephalogram spectrograms under general anesthesia maintained with sevoflurane 702 and propofol 704. In these spectrograms, frequencies are arranged along the y-axis, and time is along the x-axis, and power is indicated by color on a decibel (dB) scale. Selected 10-second epochs of

TABLE 3
Adjunct Medications Administered*

| | Sevoflurane | Propofol |
|---|---|---|
| | (n = 30) | (n = 30) |
| Midazolam (mg), mean (±SD) | 1.9 (0.4) | 1.9 (0.7) |
| | (n = 23) | (n = 14) |
| Fentanyl (mcg), mean (±SD) | 210 (80) | 192 (97) |
| | (n = 28) | (n = 24) |
| Propofol-post induction (mg), mean (±SD) | 20.0 | 55 (27) |
| | (n = 1) | (n = 12) |
| Remifentanil (mcg/kg/hr), mean (±SD) | (n = 0) | 0.09 (0.04) |
| | | (n = 24) |
| Hydromorphone (mg), mean (±SD) | 0.74 (0.53) | 0.6 (0.3) |
| | (n = 8) | (n = 6) |
| Keterolac (mg), mean (±SD) | (n = 0) | 30.0 |
| | | (n = 1) |
| Morphine (mg) | 5.0 | (n = 0) |
| | (n = 1) | |
| Neuromuscular blocker, n (%) | 27 (90.0) | 30 (100) |

*Medications administered from beginning of anesthetic until end of selected epoch.
hr, hour; kg, kilogram; mcg, microgram; mg, milligram; SD, standard deviation.

raw encephalogram signals from time-points encompassed exhibited similar signals in the 0.1-1 Hz, 1-4 Hz, 4-8 Hz and 8-14 Hz bandpass filtered frequency range. The spectrograms were computed using the multitaper method, implemented using the Chronux toolbox. Group-level spectrograms were also computed by taking the median across all patients. The spectrum for the selected electroencephalogram epochs were also computed.

The resulting power spectra were then averaged for all epochs, and 95% confidence intervals were computed via multitaper-based jackknife techniques. The spectral analysis parameters were: window length T=2 s with 0 s overlap, time-bandwidth product TW=3, number of tapers K=5, and spectral resolution of 3 Hz. The peak power, and its frequency, was also estimated for the frontal alpha oscillation for each individual subject. Results were averaged across subjects to obtain the group-level peak power and frequency for these oscillations.

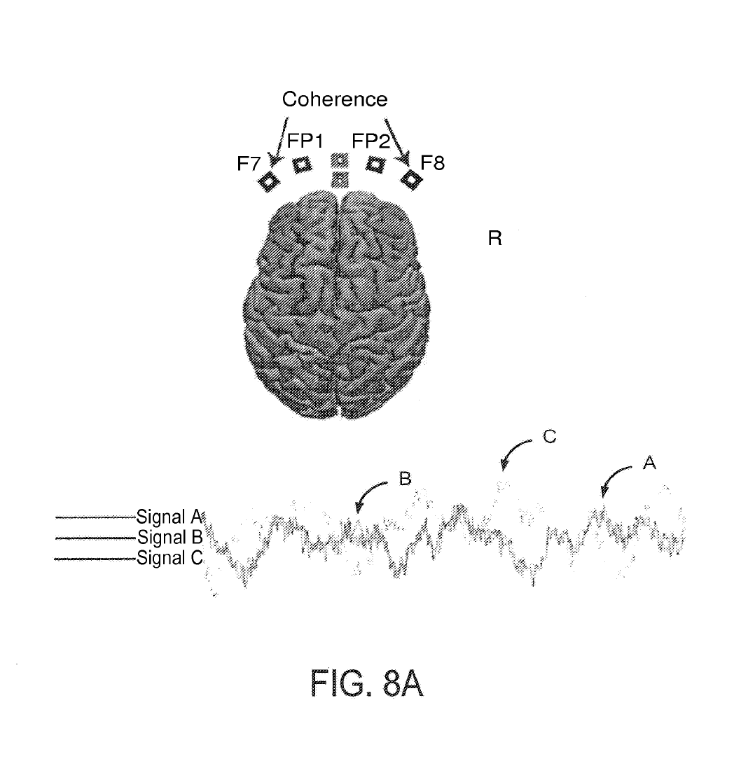

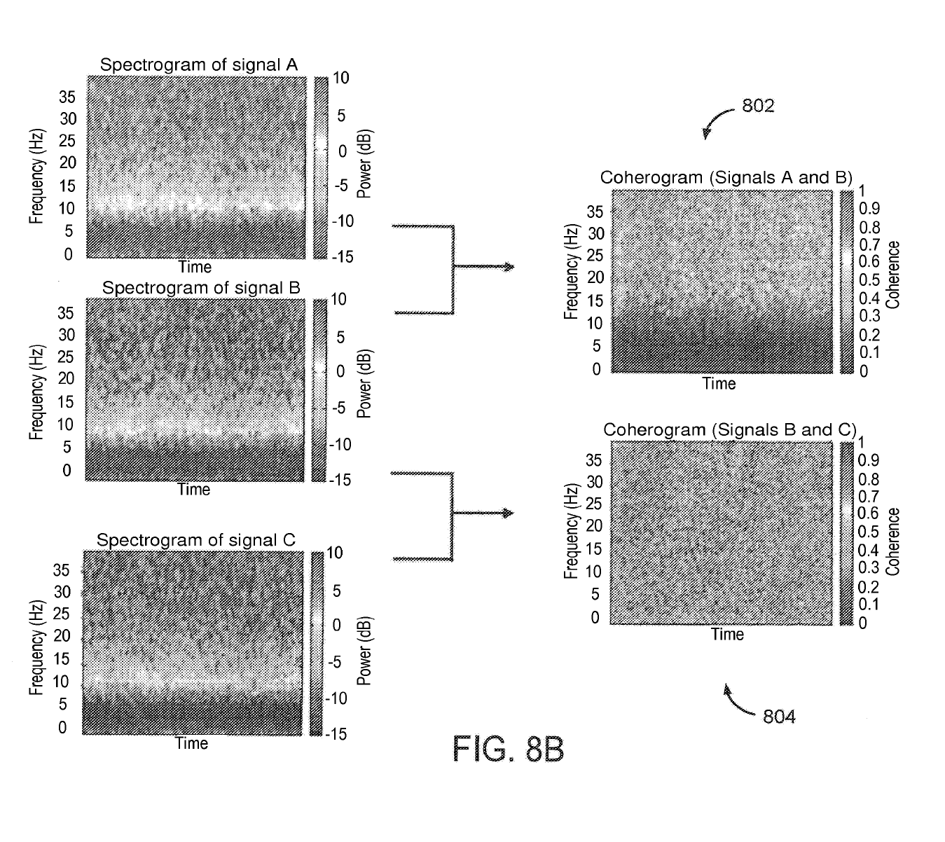

Coherence quantifies the degree of correlation between two signals at a given frequency. It is equivalent to a correlation coefficient indexed by frequency, whereby a coherence of 1 indicates that two signals are perfectly correlated at that frequency, while a coherence of 0 indicates that the two signals are uncorrelated at that frequency. The coherence function between two signals x and y is defined as:

$${C}_{xy} ( f ) = \frac{\vert {s}_{xy} ( f ) \vert}{\sqrt{{s}_{xx} ( f ) {s}_{xy} ( f )}}$$

where S_{xy}(f) is the cross-spectrum between the signals x(t) and y(t), Sₓₓ(f) is the power spectrum of the signal x(t), and S_{yy}(f) is the power spectrum of the signal y(t). Similar to the spectrum and spectrogram, the coherence can be estimated as time-varying quantity called the coherogram. To obtain estimates of coherence, coherograms were computed between two frontal electroencephalogram electrodes F7 and F8 (FIG. 8A) using the multitaper method, implemented in the Chronux toolbox. To illustrate how the coherogram quantifies relationships between signals, and how this is distinct from the spectrogram, a simulated data example was devised. Specifically, FIG. 8A shows time domain traces from three simulated oscillatory signals, two of which are highly correlated (signal A and signal B), and one which is uncorrelated with the other two (signal C). FIG. 8B shows the spectrograms for these signals, and resulting coherograms for signal pairs A-B, indicated by label 802, and B-C, indicated by label 804. As appreciated from FIG. 8B, all three signals have nearly identical spectrograms, by construction, but the coherence between the signals is very different, reflecting the presence or absence of the visible correlation evident in the time domain traces. The coherogram also indicates the frequencies over which two signals are correlated. In the coherogram labeled 802, signals A and B are correlated at frequencies below approximately 20 Hz. This example shows how the coherogram characterizes the correlation between two signals as a function of frequency. The coherence can be interpreted similarly.

Group-level coherograms were also computed by taking the median across the patients studied. Similarly, coherence was calculated for the selected electroencephalogram epochs. The resulting coherence estimates were averaged for all epochs, and 95% confidence intervals were computed via multitaper-based jackknife techniques. The coherence analysis parameters were: window length T=2 s with 0 s overlap, time-bandwidth product TW=3, number of tapers K=5, and spectral resolution of 2 W=3 Hz. The peak coherence, and its frequency, was estimated for the frontal alpha oscillation for each individual subject. An average was then computed across subjects to obtain the group-level peak coherence and frequency for these oscillations.

To compare spectral and coherence estimates between groups, jackknife-based methods were used, namely two-group test for spectra, and the two-group test for coherence, as implemented in the Chronux toolbox routine. This method accounts for the underlying spectral resolution of the spectral and coherence estimates, and considers differences to be significant only if they are present for contiguous frequencies over a frequency band wider than the spectral resolution 2 W. Specifically, for frequencies f>2 W, the null hypothesis was rejected only if the test statistic exceeded the significance threshold over a contiguous frequency range 2 W. For frequencies 0≦f≦2 W, to account for the properties of multitaper spectral estimates at frequencies close to zero, the null hypothesis was rejected only if the test statistic exceeded the significance threshold over a contiguous frequency range from 0 to max (f,W)≦2 W. A significance threshold of p<0.001 was selected for comparisons between the two groups.

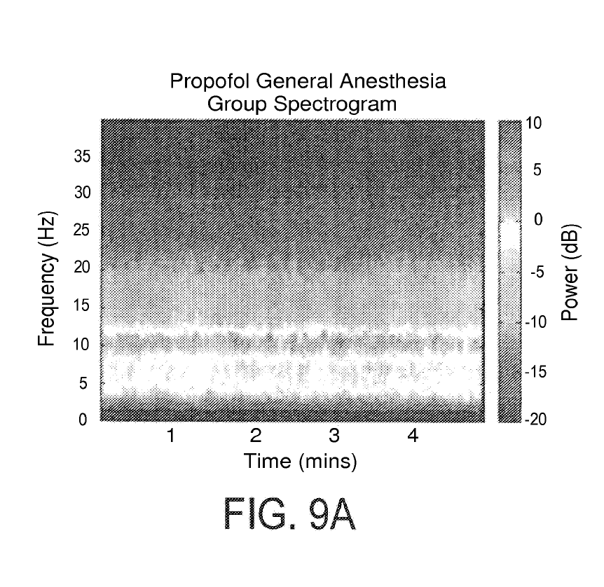

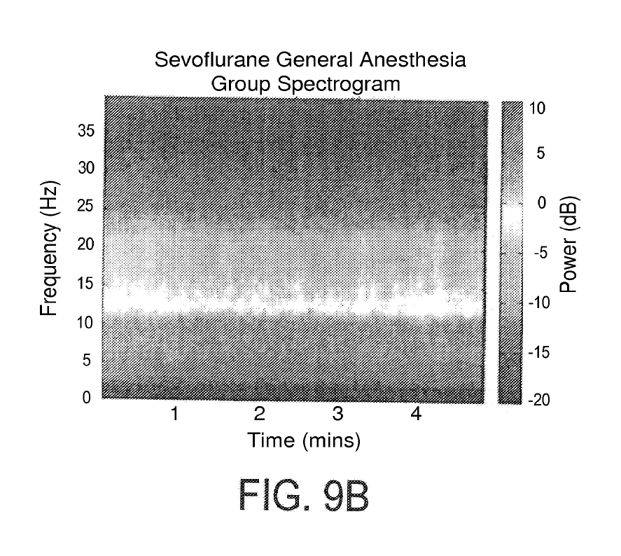

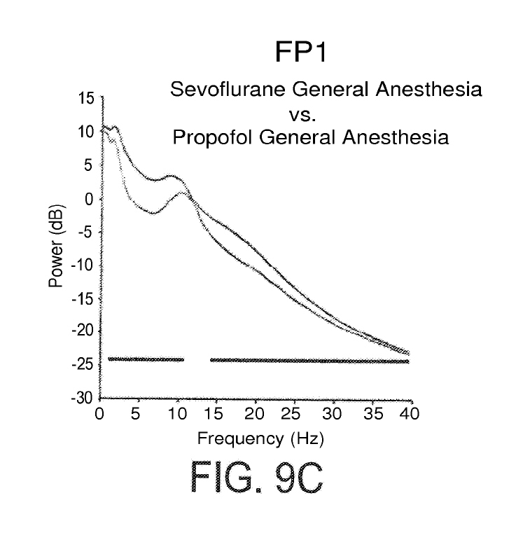

Similarities and differences in the spectrograms of the sevoflurane and propofol general anesthesia groups were observed, illustrated in FIGS. 9A, 9B. Both spectrograms were similarly characterized by large alpha band power. However, sevoflurane elicited higher power across the theta (4-8 Hz) and beta (12-25 Hz) frequency ranges. Sevoflurane general anesthesia electroencephalogram power exhibited an alpha oscillation peak (mean±std; peak frequency, 9.2 Hz±0.84; peak power, 4.3 dB±3.5) that was only slightly different from the propofol general anesthesia alpha oscillation peak (peak frequency, 10.3 Hz±1.1; peak power, 2.1 dB±4.3). The electroencephalogram spectrum was compared between these two groups finding significant differences in power across most frequencies between 0.4 and 40 Hz. Sevoflurane exhibited increased electroencephalogram power across a range of frequencies except at slow oscillations (<0.4 Hz) and the propofol alpha oscillation peak (FIG. 9C; 0.4-11.2 Hz, 14.7-40 Hz; P<0.001, two-group test for spectra). As illustrated in FIG. 9C, compared to propofol-induced unconsciousness, sevoflurane-induced unconsciousness was characterized by larger theta and beta oscillation power, and similar slow and alpha oscillation power.

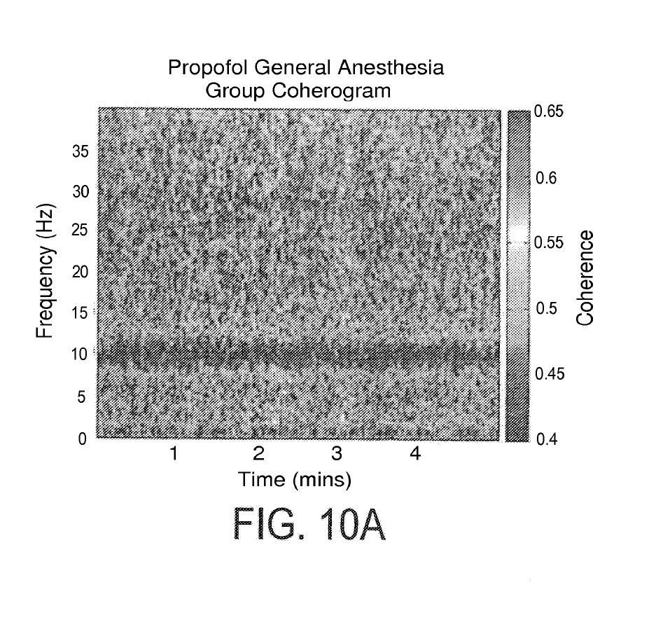

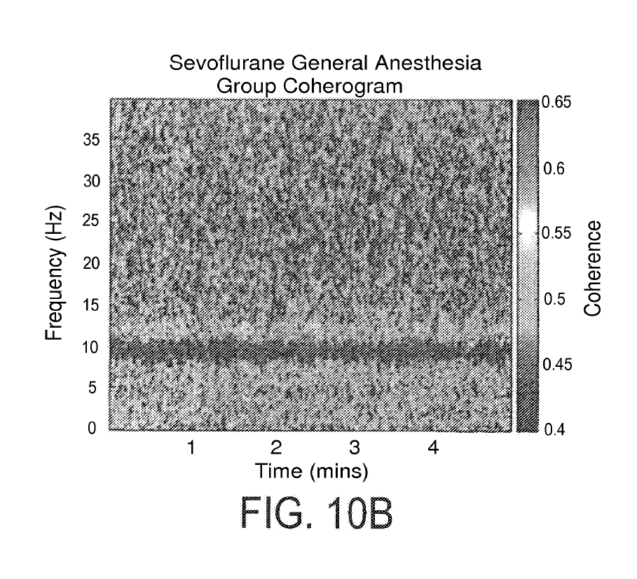

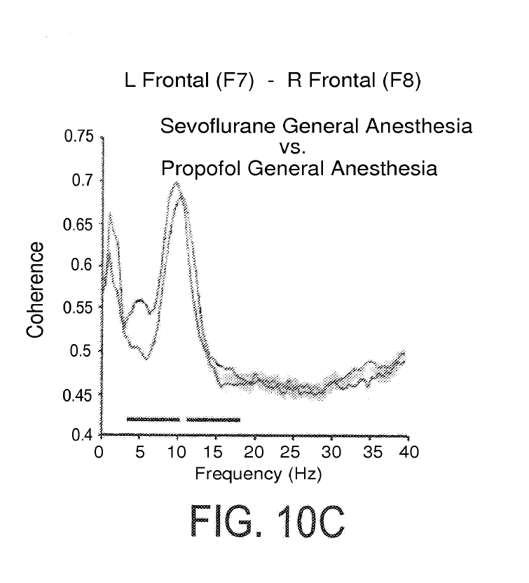

Similarities and differences were also observed in coherograms of the sevoflurane and propofol general anesthesia groups, illustrated in FIGS. 10A and 10B. Both coherograms were similarly characterized by alpha band coherence, and the absence of slow oscillation coherence. However, the sevoflurane group coherogram also showed a coherence peak within the theta frequency range that was not evident in the propofol general anesthesia group (FIGS. 10A and 10B; peak frequency, 4.9 Hz±0.6; peak coherence, 0.58±0.1). Sevoflurane general anesthesia electroencephalogram coherence exhibited an alpha oscillation peak (peak frequency, 9.8 Hz±0.91; peak coherence, 0.73±0.1) that was very similar to propofol general anesthesia alpha oscillation peak (peak frequency, 10.2 Hz±1.3; peak coherence, 0.71 dB±0.1). Comparing the electroencephalogram coherence between these two groups it was found that the sevoflurane and propofol coherence were qualitatively similar, showing a strong alpha peak, and lower slow oscillation peak. Sevoflurane exhibited increased electroencephalogram coherence across a range of theta and alpha frequencies (FIG. 10C; 3.41-10.7 Hz; two-group test for coherence, P<0.001) while propofol exhibited increased electroencephalogram coherence across a slightly different range of alpha and beta frequencies (FIG. 10C; 11.7-19.5 Hz; two-group test for coherence, P<0.001). As illustrated in FIG. 10C, sevoflurane and propofol general anesthesia were characterized by coherent frontal alpha oscillations with very similar peak frequencies and coherence values. However, sevoflurane also exhibited a coherent theta oscillation peak.

Sevoflurane- and propofol-induced electroencephalogram signatures appeared similar. These findings may be summarized as follows: (i) Similar to propofol-induced frontal alpha oscillations, sevoflurane was characterized by coherent alpha oscillations with similar maximum power and coherence occurring at −10-12 Hz; (ii) Also similar to propofol, sevoflurane was associated with slow oscillations at frequencies <1 Hz; (iii) In contrast to propofol, sevoflurane was associated with increased power and coherence in the theta band.

The similarities in sevoflurane- and propofol-induced electroencephalogram dynamics are consistent with the notion that similar GABAergic neural circuit mechanisms are involved. T his suggests that sevoflurane, like propofol, may also induce highly structured thalamocortical oscillations that interfere with cortical information processing, as well as slow oscillations that fragment cortical activity. Preliminary studies suggest that these electroencephalogram signatures are also representative of the ether derivatives, isoflurane and desflurane, suggesting that oscillatory patterns may be used as electroencephalogram signatures of general anesthesia induced loss of consciousness. It is important to note that intracortical mechanisms may also be necessary for the generation and propagation of coherent oscillations.

The coherent theta oscillations (approximately at 5 Hz) characteristic of sevoflurane anesthesia, have not been previously reported. Considering on the possible significance of these theta oscillations, it is noteworthy that pathological theta oscillations have been linked to dysfunction of low-threshold T-type calcium channels in thalamic neurons, leading to a thalamocortical dysrhythmia. Volatile anesthetics have been reported to modulate T-type calcium channels at clinically relevant concentrations in the dorsal root ganglia, hippocampal and thalamic relay neurons. These parallels lead to the hypothesize that sevoflurane-induced theta oscillations may be indicative of profound thalamic deafferentation. If true, this electroencephalogram signature along with those of slow and alpha oscillations would be useful to monitor depth of anesthesia in real-time.

Findings described herein demonstrate that propofol and sevoflurane, despite quantitative differences in the electroencephalogram power spectrum, both exhibited highly coherent frontal alpha oscillations that have been associated with entrainment of thalamocortical communications. However, sevoflurane also exhibited a theta-band coherence that was not present under propofol. Coherent theta oscillations were not generally present in the awake eyes closed state, leading to the conclusion that this coherence signature was sevoflurane induced. Also, such similarities and differences in electroencephalogram spectra and coherences were also observed in data recorded during routine care of patients undergoing a variety of surgical procedures, and under different co-administered medications, suggesting that these effects are robust.

The electroencephalogram recordings analyzed herein were obtained from frontal channels. As a result, the analysis described herein did not examine anterior-posterior connectivity, which has been reported as other cortical dynamics underlying anesthesia induced unconsciousness. Also, since this study was performed in the clinical setting with concomitant administration of opioids, there were fewer detailed characterizations of changing behavior and consciousness during controlled induction and emergence, limiting inferences to a clinically unconscious state. It is envisioned that future studies employing high-density electroencephalogram and behavioral tasks will allow analysis of connectivity and phase-amplitude coupling under sevoflurane and other inhaled anesthetics and their relation to varying degrees of consciousness. In summary, the present analysis suggests a potential shared GABAergic mechanism for propofol and sevoflurane at clinically-relevant doses. Furthermore, it details electroencephalogram signatures that can be used to identify and monitor the shared and differential effects of anesthetic agents, providing a foundation for future analyses, as well as an approach for characterizing and identifying one or more administered drug.

The present invention has been described in terms of one or more preferred embodiments, and it should be appreciated that many equivalents, alternatives, variations, and modifications, aside from those expressly stated, are possible and within the scope of the invention.

1. A system for evaluating an effectiveness of one or more drugs administered to a subject, the system comprising;
an input configured to receive neurophysiological data acquired from a subject;
a processor programmed to at least:
i) analyze the neurophysiological data to generate signatures indicative of brain states induced by one or more drugs administered to the subject;
ii) correlate the generated signatures with a database comprising information associated with a plurality of drug profiles;
iii) determine, using the information, a molecular activity profile for the one or more drugs;
iv) generate, using the molecular activity profile, a report indicative of the effectiveness of the one or more drugs; and
an output for displaying the report.

2. The system of claim 1, wherein the system further includes one or more sensors arranged configured to acquire the neurophysiological data.

3. The system of claim 1, wherein the neurophysiological data includes at least one of electroencephalogram (“EEG”) data, electromyography (“EMG”) data, behavioral data, respiratory data, blood flow data, cardiac data, and galvanic skin response data.

4. The system of claim 1, wherein the processor is configured to perform at least one of a waveform analysis, a spectral analysis, a coherence analysis, a phase analysis, and a synchrony analysis.

5. The system of claim 1, wherein in analyzing the acquired neurophysiological data the processor is further configured to receive an indication of clinical states targeted by the one or more drugs.

6. The system of claim 5, wherein the clinical states include at least one of a sedation, a general anesthesia, a recovery from depression, and a suppression of epileptic activity.

7. The system of claim 1, wherein the processor is further configured to assemble the neurophysiological data into a frequency representation using a multitaper technique.

8. The system of claim 1, wherein the processor is further configured to generate simulation data for the one or more drugs based on the molecular activity profile determined at step iii).

9. The system of claim 8, wherein the process is further configured to validate the molecular activity profile by comparing the simulation data and the neurophysiological data.

10. The system of claim 1, wherein the processor is further configured to identify the one or more drugs based on the molecular activity profile determined at step iii).

11. A method for evaluating an effectiveness of a drug administered to a subject, the method comprising:
a) receiving neurophysiological data acquired from a subject under an administration of a drug;
b) analyzing the neurophysiological data to generate signatures indicative of brain states induced by the drug;
c) correlating the generated signatures with a database comprising information associated with a plurality of drug profiles;
d) determining, using the information, a molecular activity profile for the drug; and
e) generating a report indicative of the effectiveness of the drug using the molecular activity profile.

12. The method of claim 11, wherein the method further comprises acquiring the neurophysiological data using one or more sensors arranged about the subject.

13. The method of claim 11, wherein the neurophysiological data includes at least one of EEG data, electromyography (“EMG”) data, behavioral data, respiratory data, blood flow data, cardiac data, and galvanic skin response data.

14. The method of claim 11, wherein the method further comprises performing at least one of a waveform analysis, a spectral analysis, a coherence analysis, a phase analysis, and a synchrony analysis.

15. The method of claim 11, wherein in analyzing the acquired neurophysiological data at step b) includes receiving an indication of one or more clinical states targeted by the drug.

16. The method of claim 15, wherein the one or more clinical states includes at least one of a sedation, a general anesthesia, a recovery from depression, an enhancement of cognition, an impairment of cognition, and a suppression of epileptic activity.

17. The method of claim 1, wherein the method further comprises assembling the neurophysiological data into a frequency representation using a multitaper technique.

18. The method of claim 1, wherein the method further comprises generating simulation data for the drug based on the molecular activity profile determined at step d).

19. The method of claim 18, wherein the method further comprises comparing the simulation data and the neurophysiological data to validate the molecular activity profile of the drug.

20. The method of claim 11, wherein the method further comprises identifying the drug based on the molecular activity profile determined at step d).

21. The method of claim 11, wherein the method further comprises modifying the drug based on the effectiveness determined.

22. The method of claim 11, wherein the method further comprises repeating steps a) through e) for a plurality of drugs and drug doses to select drug candidates for achieving one or more targeted clinical states.

23. A method for characterizing a neuroactive drug administered to a subject, the method comprising:
a) acquiring neurophysiological data using one or more sensors arranged about a subject having received a neuroactive drug;
b) generating, using the neurophysiological data, signatures indicative of brain states induced by the neuroactive drug;
c) identifying, using the generated signatures, molecular receptor actions associated with the induced brain states; and
d) generating a report characterizing the neuroactive drug using the identified molecular receptor actions.

24. The method of claim 23, wherein the method further comprises performing at least one of a waveform analysis, a spectral analysis, a coherence analysis, a phase analysis, and a synchrony analysis.

25. The method of claim 23, wherein the method further comprises comparing the generated signatures with a database comprising information associated with a plurality of drug profiles.

26. The method of claim 23, wherein the neurophysiological data is associated with at least one of a neural circuit, a neural system, a neuronal network.

27. The method of claim 23, wherein the method further comprises receiving information associated with at least one of a patient profile, and an administration of the neuroactive drug.

28. The method of claim 23, wherein the method further comprises identifying a dominant receptor affinity based on a dose of the neuroactive drug.
